(19) United States
(12) Patent Application Publication
(10) Pub. No.: US 2012/0259313 A1
(43) Pub. Date: Oct. 11, 2012

(54) APPARATUS AND METHODS FOR LIMITING PRESSURE AND FLOW WITHIN A MEDICAL INJECTOR

(75) Inventors: John F. Krumme, Woodside, CA (US); Scott Heneveld, Whitmore, CA (US)

(73) Assignee: NORDSON CORPORATION, Westlake, OH (US)

(21) Appl. No.: 13/500,500

(22) PCT Filed: Dec. 14, 2010
(86) PCT No.: PCT/US10/60225

§ 371 Date: Jun. 15, 2012

Related U.S. Application Data
(60) Provisional application No. 61/286,497, filed on Dec. 15, 2009.

(51) Int. Cl. A61M 5/34

(52) U.S. Cl. 604/506; 604/240

(57) ABSTRACT

A medical injector (200) includes a medicament container (210) including a main lumen (214) and a reduced-diameter protrusion (220), a needle assembly (230) coupled to the protrusion (220), and a flow control member (250). The medicament container (210) is configured to contain a medicament, such as, for example, a dermal filler. The flow control member (250) is disposed within the protrusion (220) of the medicament container (210), and includes a flow control lumen (254) configured to control a pressure or a flow of the medicament. Thus, the flow control member (250) reduces decoupling forces applied to the needle assembly (230) by the flow of medicament. Alternatively, the flow control member (250) may move from a first position allowing flow from the main lumen (214) to the needle assembly (230) to a second position blocking flow from the medicament container (210) when the needle assembly (230) decouples from the protrusion (220).

### CROSS-REFERENCE TO RELATED APPLICATION

This application claims the priority of U.S. Provisional Patent Application Ser. No. 61/286,497, filed on Dec. 15, 2009 (pending), the disclosure of which is incorporated by reference herein.

### BACKGROUND

The invention relates generally to medical injectors and, more particularly, to apparatus and methods for limiting flow of a medicament within a medical injector.

Some known medical injectors, such as, for example, syringes, can be used to inject medicaments, therapeutic agents, and/or compositions into a body of a patient. For example, some known medical injectors can be used to inject a dermal filler into the body to augment soft tissue portions of the body. For example, known medical injectors can be used to inject high viscosity compositions adjacent the urinary sphincter muscle to increase the volume of the tissue within the urinary tract to treat urinary incontinence. Known medical injectors can also be used to inject high viscosity compositions into the skin to change the contour of and/or increase the volume of the skin. For example, known high viscosity compositions can be injected within facial skin to remove wrinkles, treat scars or the like. Such known medical injectors can use a small-bore needle to minimize pain and trauma to the patient. The needle can be coupled to the medical injector, for example, by threaded coupling, such as, for example, a Luer fitting.

When known medical injectors are used to inject high viscosity compositions via a small-bore needle, the pressure of the composition within the medical injector can be relatively high. In various instances, for example, the pressure of the composition in the medical injector can be at low levels or up to about 300 pounds per square inch. Accordingly, in certain circumstances, the flow and/or pressure of the medicament within the needle can be irregular due to the high pressure and/or pressure fluctuations within the medical injector that can result from the manual force applied by a medical practitioner during the injection event. In certain circumstances, the pressure of the medicament within the needle can cause the coupler and/or the needle to fail, thereby causing the needle to become detached from the medical injector. Such needle detachment can result in discomfort for the patient, injury to the patient or nearby personnel, contamination of filler and/or waste of filler.

Thus, a need exists for a medical injector that can limit and/or control the flow of the medicament, therapeutic agent and/or composition being injected. A need also exists for improved methods of limiting the pressure and/or flow of the medicament, therapeutic agent and/or composition being injected if needle detachment and/or potential needle detachment occurs.

### SUMMARY

Medical injectors and methods are described herein. In one embodiment, a medical injector includes a medicament container and a flow control member. The medicament container is configured to contain a medicament, such as, for example, a dermal filler. The medicament container includes a reduced-diameter protrusion extending distally from a main lumen and an internal shoulder between the main lumen and the protrusion. The protrusion of the medicament container is configured to be coupled to a needle assembly. The flow control member is disposed at least partially within the protrusion. The flow control member includes a flow control lumen configured to control a flow of the medicament from the medicament container to the needle assembly. The flow control member may therefore reduce decoupling forces applied to the needle assembly by the flow of the medicament.

In one embodiment, the flow control lumen of the flow control member includes a diameter or a length selected such that the flow control member reduces the pressure or the flow of the medicament into the needle assembly. The reduced pressure or flow of the medicament reduces the decoupling forces applied to the needle assembly by the flowing medicament.

In another embodiment, the flow control member is positioned adjacent to the protrusion of the medicament container. The flow control member is configured to move from a first position to a second position when forces applied by the medicament to the needle assembly decouple the needle assembly and the protrusion. In the first position, medicament may flow from the main lumen to the needle assembly. In the second position, the flow control member blocks flow of medicament from the medicament container.

In another embodiment, a method of dispensing medicament into a patient with a medical injector includes inserting a needle of the medical injector into the patient. The method also includes actuating a flow of medicament from a medicament container to a needle assembly including the needle. The method also includes applying forces to the needle assembly with the medicament until the needle assembly decouples from the protrusion. When the needle assembly becomes decoupled from the medicament container, the method may include blocking the flow of the medicament from the medicament container with the flow control member.

Various other features, details and advantages of the invention will become more readily apparent to those of ordinary skill in the art upon review of the following detailed description of the illustrative embodiments, taken in conjunction with the accompanying drawings.

### BRIEF DESCRIPTION OF THE DRAWINGS

FIG. 1 is a schematic illustration of a medical injector according to one embodiment of the invention.

FIG. 2 is a perspective view of a medical injector according to another embodiment.

FIG. 3 is a cross-sectional view of a portion of the medical injector shown in FIG. 2 taken along line X-X in FIG. 2.

FIG. 4 is a perspective view of a flow control member of the medical injector shown in FIG. 2.

FIG. 5 is a cross-sectional view of a portion of a medical injector according to another embodiment.

FIG. 6 is a perspective view of a flow control member of the medical injector shown in FIG. 5.

FIG. 7 is a cross-sectional view of a portion of a medical injector according to another embodiment.

FIG. 8 is a perspective view of a seal member of the medical injector shown in FIG. 7.

FIG. 9 is a cross-sectional view of a portion of a medical injector according to another embodiment.

FIG. 10 is a schematic illustration of a medical injector according to another embodiment, in a first configuration.

FIG. 11 is a schematic illustration of the medical injector shown in FIG. 10 in a second configuration.

FIG. 12 is a schematic illustration of a medical injector according to another embodiment, in a first configuration.

FIG. 13 is a schematic illustration of the medical injector shown in FIG. 12 in a second configuration.

FIG. 14 is a cross-sectional view of a portion of a medical injector according to another embodiment, in a first configuration.

FIG. 15 is a cross-sectional view of the medical injector shown in FIG. 14 in a second configuration.

FIG. 16 is a perspective view of a flow control member of the medical injector shown in FIGS. 14 and 15.

FIG. 17 is a flow chart of a method according to one embodiment of the invention.

FIG. 18 is a flow chart of a method according to another embodiment.

### DETAILED DESCRIPTION

In some embodiments, an apparatus includes a medicament container and a flow control member. The medicament container, which can be a glass syringe, is configured to contain a medicament (e.g., a dermal filler). The medicament container has a distal end portion and a proximal end portion. The distal end portion of the medicament container is configured to be coupled to a needle assembly. In some embodiments, for example, the distal end portion of the medicament container can be coupled to the needle assembly via a twist-on Luer fitting (e.g., a Luer-Lok™ fitting). The flow control member is disposed within the distal end portion of the medicament container. The flow control member is configured to control a flow of the medicament through and/or a pressure of the medicament within the needle assembly.

In some embodiments, an apparatus includes a medicament container and a seal member. The medicament container has a distal end portion and a proximal end portion, and is configured to be coupled to a needle assembly. The distal end portion of the medicament container includes a protrusion configured to be disposed within a needle hub of the needle assembly such that at least a portion of the protrusion and the needle hub form a first substantially fluid-tight seal. The seal member has a proximal end portion and a distal end portion. At least the proximal end portion of the seal member is disposed within the distal end portion of the medicament container. In some embodiments, for example, the seal member can be entirely disposed within the medicament container. The distal portion of the seal member and at least one of the needle hub and a needle of the needle assembly form a second substantially fluid-tight seal.

In some embodiments, an apparatus includes a medicament container, a coupler, and a flow member. The medicament container has a distal end portion and a proximal end portion, and is configured to contain a medicament. The coupler is configured to couple a needle assembly to the distal end portion of the medicament container. The flow control member is coupled to the distal end portion of the medicament container. The flow control member is configured to substantially fluidically isolate the medicament container from the needle assembly when the coupler fails to maintain a position of the needle assembly relative to the distal end portion of the medicament container. Similarly stated, the flow control member is configured to limit the flow of the medicament from the medicament container to the needle assembly when the needle assembly moves relative to the distal end portion of the medicament container.

In some embodiments, a method includes inserting a needle of a syringe into a body of a patient. The syringe is actuated such that a medicament is conveyed from a medicament container of the syringe into the body of the patient through the needle. The needle is fluidically isolated from the medicament container when the needle becomes decoupled from the medicament container during the actuation of the syringe.

As used in this specification, the words “proximal” and “distal” refer to the direction closer to and away from, respectively, an operator (e.g., surgeon, physician, nurse, technician, etc.) who would use a medical injector during a procedure. For example, the end of a medical injector first to contact and/or be inserted into the patient's body would be the distal end, while the opposite end of the medical injector (e.g., the end of the medical injector being operated by the operator) would be the proximal end of the medical injector.

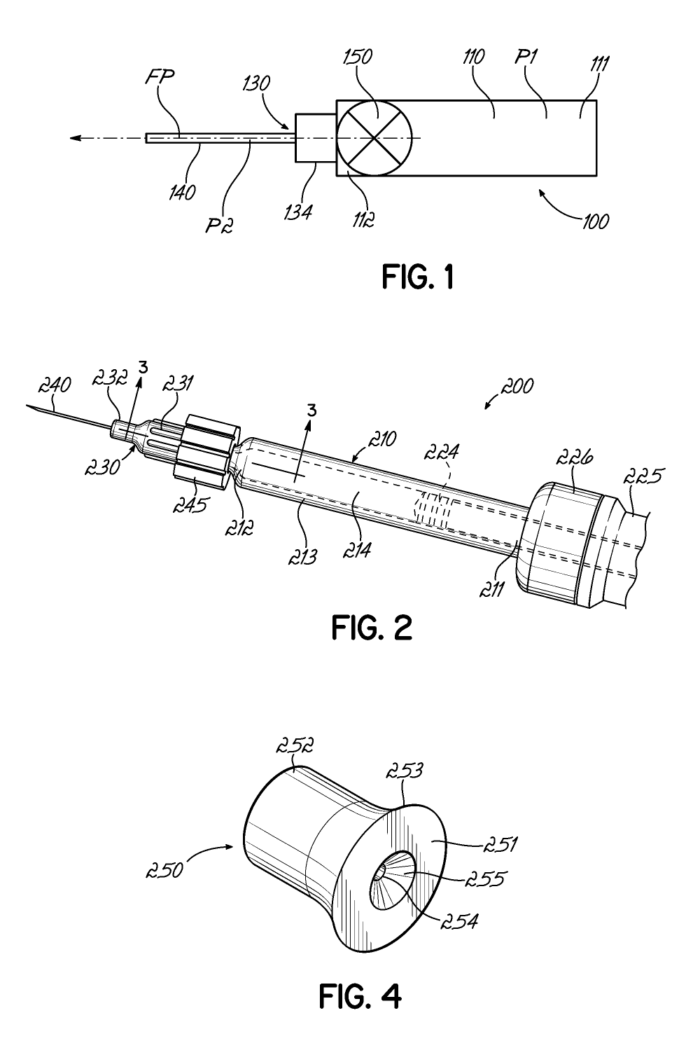

FIG. 1 is a schematic illustration of a medical injector 100 according to an embodiment. The medical injector 100 includes a medicament container 110, a needle assembly 130 and a flow control member 150. The medicament container 110 has a proximal end portion 111 and a distal end portion 112. The medicament container 110 is configured to contain a medicament (not shown in FIG. 1), such as, for example, a dermal filler, a sub-dermal filler, a therapeutic substance for mesotherapy or the like. The medicament container 110 can include a plunger (not shown in FIG. 1) configured to displace the medicament within the medicament container 110. Similarly stated, the medicament container 110 can include a plunger configured to exert a force on the medicament thereby causing the medicament to be injected from the medicament container 110 through the needle assembly 130 along a medicament flow passageway FP (shown as a dashed line in FIG. 1).

The needle assembly 130 includes a needle 140 and a hub 134. The needle 140 can be, for example, a small-bore needle (e.g., a needle having a bore size less than or equal to that of a 27 gauge needle) configured to convey the medicament from the medicament container 110 to the patient's body. The needle 140 and the hub 134 are collectively coupled to the distal end portion 112 of the medicament container 110 such that the needle 140 is in fluid communication with the medicament container 110. The needle assembly 130 can be coupled to the medicament container 110 by any suitable coupler (e.g., a Luer connector) that provides a substantially fluid-tight seal (i.e., a seal that substantially prevents a liquid and/or a gas from passing therethrough) between the needle assembly 130 and the medicament container 110. Moreover, such a coupler can be configured to maintain a position of the needle assembly 130 relative to the distal end 112 of the medicament container 110.

The flow control member 150 is disposed within the distal end portion 112 of the medicament container 110 and is configured to control a flow of the medicament from the medicament container 110 to the needle assembly 130. Similarly stated, the flow control member 150 is configured to control a pressure and/or a flow rate of the medicament along the flow passageway FP during an injection event. In some embodiments, for example, the flow control member 150 can produce a pressure drop within the flow passageway FP during an injection event. More particularly, in some embodiments, the flow control member 150 can cause the pressure of the medicament to be reduced from a first pressure P1 at a first location along the flow passageway FP (within the medicament container 110) to a second pressure P2 at a second location along the flow passageway FP (within the hub 134 and/or the needle 140). In this manner, the flow control member 150 can limit the pressure P2 within the hub 134 and/or the needle 140. For example, in some embodiments, the flow control member 150 can be configured to prevent the pressure P2 within the hub 134 and/or the needle 140 from exceeding a predetermined value.

Although the flow control member 150 is described above as controlling the pressure P2 and/or the flow of the medicament within the needle hub 134 and/or the needle 140, in some embodiments, the flow control member 150 can control the rate of change of the pressure P2 and/or the flow of the medicament within the needle hub 134 and/or the needle 140. Said another way, in some embodiments, the flow control member 140 can limit the temporal fluctuation of the flow and/or the pressure P2 of the medicament within the needle hub 134 and/or needle 140. Similarly stated, in some embodiments, the flow control member 150 can reduce and/or dampen the temporal variance of the flow and/or the pressure P2 of the medicament within the needle hub 134 and/or needle 140. In this manner, the medicament can be injected from the medical injector 100 in a controlled, continuous and/or smooth fashion. Moreover, by limiting the peak value of the pressure P2, the likelihood of failure of the coupling between the needle assembly 130 and the medicament container 110 can be reduced. Similarly stated, by limiting the peak value of the pressure P2, the likelihood that the needle assembly 130 will be inadvertently decoupled from the medicament container 110 can be reduced.

The flow control member 150 can include any suitable mechanism for controlling the flow and/or pressure of the medicament as described above. For example, in some embodiments, the flow control member 150 can include a dynamic device, such as, for example, a movable valve configured to control the flow and/or the pressure of the medicament. Such a valve can be, for example, a ball valve, a poppet valve, a check valve or the like. In other embodiments, the flow control member 150 can include a static device, such as, for example, an orifice configured to control the flow and/or the pressure of the medicament.

FIGS. 2-4 show a medical injector 200 according to another embodiment. The medical injector 200 includes a medicament container 210, a needle assembly 230, an actuator 225, and a flow control member 250. The medicament container 210, which can be, for example, a commercially-available glass syringe, has a proximal end portion 211 and a distal end portion 212. The medicament container 210 includes a side wall 213 that defines a lumen 214. The lumen 214 of the medicament container 210 is configured to contain a medicament, such as, for example, a dermal filler, a sub-dermal filler, a therapeutic substance for mesotherapy or the like. A plunger 224 (shown in phantom in FIG. 2) is movably disposed within the lumen 214 of the medicament container 210 such that movement of the plunger 224 results in displacement of the medicament within the medicament container 210.

The proximal end portion 211 of the medicament container 210 is coupled to the actuator 225 via a coupler 226. In some embodiments, the coupler 226 can removably couple the actuator 225 to the medicament container 210. For example, in some embodiments, the coupler 226 can threadably couple the actuator 225 to the medicament container 210. In other embodiments, the coupler 226 can form a press-fit coupling between the actuator 225 and the medicament container 210.

The actuator 225 is configured to move the plunger 224 within the medicament container 210 such that the medicament is conveyed to the patient's body via the needle assembly 230. In some embodiments, the actuator 225 can include a source of pressurized fluid that exerts a force on the plunger 224. In some such embodiments, the actuator 225 can also include a pressure amplifier configured to receive a fluid at a first pressure and to produce a force on the plunger 224 such that a pressure of the medicament within the medicament container 210 is at a second pressure greater than the first pressure. In other embodiments, the actuator 225 can be a mechanical device configured to move the plunger 224. For example, in some embodiments, the actuator 225 can include a stepper motor configured to move the plunger 224 within the medicament container 210.

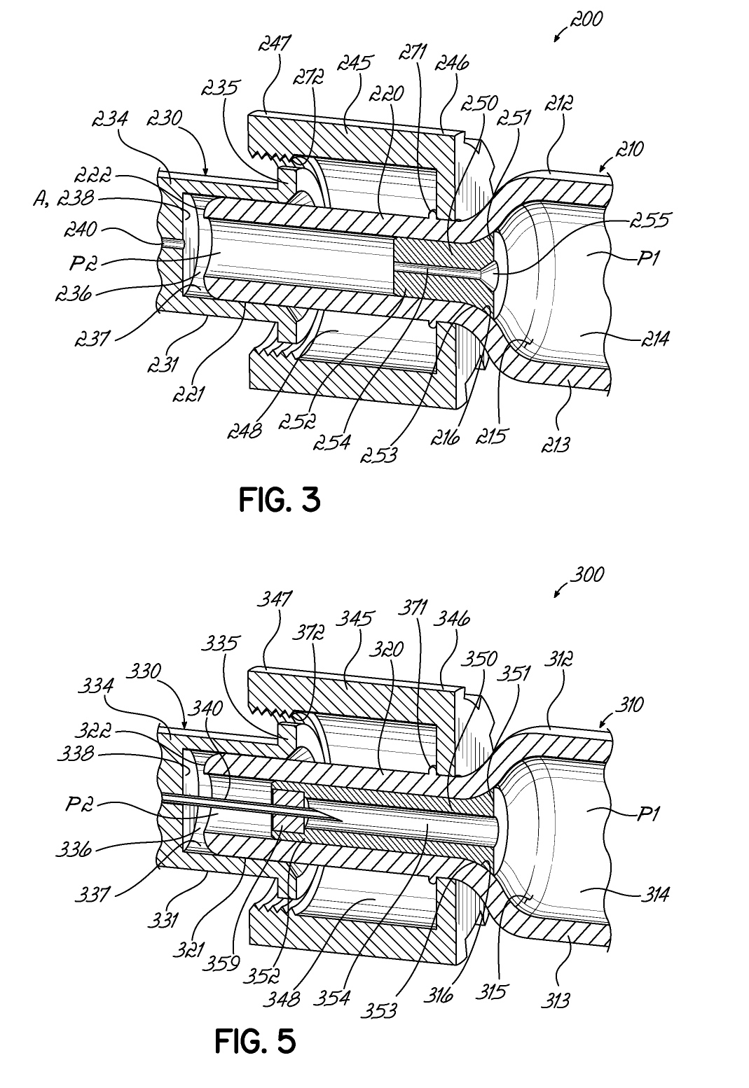

As shown in FIG. 3, the distal end portion 212 of the medicament container 210 includes a protrusion 220 having an outer surface 221 and a distal end surface 222. At least a portion of the protrusion 220 is configured to be disposed within a volume 236 defined by a needle hub 234 when the needle assembly 230 is coupled to the medicament container 210, as described in more detail herein. The protrusion 220 is tapered such that a size (e.g., an outer diameter) of the protrusion 220 decreases along a longitudinal axis of the medicament container 210 in a distal direction. In some embodiments, for example, the protrusion 220 can be tapered at an angle of approximately six degrees. The outer surface 221 of the protrusion 220 includes a lip or shoulder 271. As described in more detail below, a coupler 245 is disposed about the protrusion 220 such that the lip of the protrusion 220 limits axial movement of the coupler 245 about the protrusion 220.

The needle assembly 230 includes a needle 240 and the hub 234. The needle 240 (a portion of which is shown in FIG. 3) can be, for example, a small-bore needle (e.g., a needle having a bore size less than or equal to that of a 27 gauge needle) configured to convey the medicament from the medicament container 210 to the patient's body. As shown in FIGS. 2 and 3, the hub 234 includes a distal end portion 232 and a proximal end portion 231. The needle 240 is disposed within the distal end portion 232 of the hub 234. The proximal end portion 231 of the hub 234 includes a flange 235 and defines the volume 236. The tapered protrusion 220 of the medicament container 210 is disposed within the volume 236 of the hub 234 such that a portion of the outer surface 221 of the protrusion 220 engages a portion of an inner surface 237 of the hub 234 to produce a substantially fluid-tight seal between the medicament container 210 and the needle assembly 230. Moreover, when the tapered protrusion 220 of the medicament container 210 is disposed within the volume 236 of the hub 234, the distal end surface 222 of the protrusion 220 is spaced apart from an interior end surface 238 of the hub 234. The engagement between the outer surface 221 of the protrusion 220 and the inner surface 237 of the hub 234 limits the axial movement of the protrusion 220 within the hub 234 in the distal direction. Similarly stated, because the distal end surface 222 of the protrusion 220 does not contact the interior end surface 238 of the hub 234, the protrusion 220 is free to move in a distal direction within the coupler 245 until the outer surface 221 of the protrusion 220 contacts the inner surface 237 of the hub 234.

The coupler 245 includes a distal end portion 247, a proximal end portion 246, and defines a lumen 248 therethrough. The coupler 245 is disposed about the protrusion 220 of the medicament container 210 such that the protrusion 220 is within the lumen 248 of the coupler 245. More specifically, a portion of the proximal end portion 246 of the coupler 245 engages the lip or shoulder 271 of the protrusion 220 such that movement of the coupler 245 axially relative to the medicament container 210 is limited. Similarly stated, the proximal end portion 246 and the lip of the protrusion 220 form a snap-fit that allows the coupler 245 to rotate relative to the medicament container 210 and/or the needle assembly 230, but limits axial movement of the coupler 245 relative to the medicament container 210.

A portion of the lumen 248 of the coupler 245 includes female threads 272 configured to receive and/or engage the flange 235 of the hub 234 to threadably couple the hub 234 to the coupler 245. Although the flange 235 is shown as being spaced apart from the inner surface of the coupler 245 for purposes of clarity, when hub 234 is disposed within the lumen 248 of the coupler 245, the flange 235 is disposed within the female threads and is thus in contact with a portion of the coupler 245. In this manner, the coupler 245 is configured to maintain a position of the needle assembly 230 relative to the distal end portion 212 of the medicament container 210. Similarly stated, in this manner, the needle assembly 230 is coupled to the distal end portion 212 of the medicament container 210 via the coupler 245 such that the needle 240 is in fluid communication with the medicament container 210. The coupler 245 can be, for example, a press-fit Luer fitting (e.g., a Luer-Slip™ fitting), a twist-on Luer fitting (e.g., a Luer-Lok™ fitting) and/or the like.

The flow control member 250 includes a proximal end portion 251 and a distal end portion 252 and defines a lumen 254 therethrough. The proximal end portion 251 of the flow control member 250 includes a tapered outer surface 253. The proximal end portion 251 of the flow control member 250 defines a chamfered opening 255 in fluid communication with the lumen 254.

The flow control member 250 is disposed within the distal end portion 212 of the medicament container 210 such that the tapered outer surface 253 is engaged with a shoulder 216 defined by an inner surface 215 of the medicament container 210. In this manner, movement of the flow control member 250 within the medicament container 210 can be limited. In some embodiments, a portion of the flow control member 250 can form an interference fit within the protrusion 220 of the medicament container 210. Additionally, in some embodiments, the tapered outer surface 253 and the shoulder 216 can form a substantially fluid-tight seal. Thus, in some embodiments, when the medicament is conveyed from the medicament container 210 to the needle assembly 230, substantially all of the medicament will be conveyed via the lumen 254. Similarly stated, in some embodiments, the lumen 254 is the only flow passageway that fluidically couples the medicament container 210 and the needle assembly 230. In other embodiments, when the medicament is conveyed from the medicament container 210 to the needle assembly 230, a portion of the medicament can be conveyed via a separate flow passageway (not shown in FIG. 3) between the inner surface 215 of the medicament container and the outer surface 253 of the flow control member 250.

The lateral size (e.g., diameter) and/or the length of the lumen 254 can be any suitable size to control a flow rate of the medicament from the medicament container 210 to the needle assembly 230. More particularly, the diameter and/or the length of the lumen 254 can be selected to produce a pressure drop when the medicament flows from the medicament container 210 to the needle assembly 230 during an injection event. More particularly, in some embodiments, the flow control member 250 can cause the pressure of the medicament to be reduced from a first pressure P1 at a first location (within the medicament container 210) to a second pressure P2 at a second location (within the hub 234 and/or the needle 240). In this manner, the flow control member 250 can control the flow and/or pressure of the medicament within the needle assembly 230 during an injection event.

More particularly, the flow control member 250 can limit the pressure P2 of the medicament within the volume 236 of the hub 234 that acts upon the interior end surface 238 of the hub 234. In this manner, the flow control member 250 can limit the force acting on the hub 234 in the distal direction, thereby reducing the likelihood that the coupler 245 and/or a portion of the hub 234 (e.g., the flange 235) will fail during use. In particular, the distal force acting on the hub 234 is a function of the pressure P2 of the medicament within the volume 236 of the hub 234 and the area A of the interior end surface 238 of the hub 234, as shown in Equation (1).

(1)

F_{distal}=P2×A

Thus, by limiting the pressure P2, the distal force acting on the hub 234 is proportionally reduced.

In addition to being based on the diameter and the length of the lumen 254, the pressure drop (i.e., P1-P2) produced by the flow control member 250 is also a function of the flow rate of the medicament through the lumen 250. More particularly, the pressure drop produced by the flow control member 250, as approximated by the Bernoulli equation, is proportional to the flow rate squared. To this end, reducing the flow through the flow control member 250 lowers the fluid pressure downstream of the flow control member 250, which also lowers the distal force acting on the hub 234 as shown above. Thus, the diameter and/or the length of the lumen 254 can be selected based on a desired flow rate of medicament during an injection event.

In some embodiments, for example, the flow control member 250 can produce a pressure drop such that when the pressure P1 within the medicament container is approximately 70 p.s.i., the pressure P2 is 50 p.s.i. or less. In other embodiments, the flow control member 250 can produce a pressure drop such that when the pressure P1 is as much as approximately 100 p.s.i., the pressure P2 is 70 p.s.i. or less. In yet other embodiments, the flow control member 250 can produce a pressure drop such that when the pressure P1 is as much as approximately 150 p.s.i., the pressure P2 is 100 p.s.i. or less. In yet other embodiments, the flow control member 250 can produce a pressure drop such that when the pressure P1 is as much as approximately 250 p.s.i., the pressure P2 is 170 p.s.i. or less.

The flow control member 250 can be configured to allow any suitable flow rate of medicament through the lumen 254 and/or the needle assembly 230. For example, in some embodiments, the flow rate of the medicament through the lumen 254 and/or the needle assembly 230 can be at least approximately 0.02 cubic centimeters per minute. In other embodiments, the flow rate of the medicament through the lumen 254 and/or the needle assembly 230 can be between approximately 0.02 cubic centimeters per minute and 0.5 cubic centimeters per minute. In yet other embodiments, the flow rate of the medicament through the lumen 254 and/or the needle assembly 230 can be as much as 3 cubic centimeters per minute. In still other embodiments, the flow rate of the medicament through the lumen 254 and/or the needle assembly 230 can be greater than 3 cubic centimeters per minute.

In some embodiments, the diameter of the lumen 254 can be less than the diameter of the bore of the needle 240. For example, in some embodiments, the bore of the needle 240 can be approximately 0.191 millimeters (27 gauge needle size) and the diameter of the lumen 254 can be less than approximately 0.180 millimeters. In other embodiments, the diameter of the lumen 254 can be greater than the diameter of the bore of the needle 240. For example, in some embodiments, the bore of the needle 240 can be approximately 0.191 millimeters (27 gauge needle size) and the diameter of the lumen 254 can be greater than approximately 0.200 millimeters. In some embodiments, for example, the diameter of the lumen 254 can be approximately 0.305 millimeters. In yet other embodiments, the diameter of the lumen 254 can be approximately equal to the diameter of the bore of the needle 240.

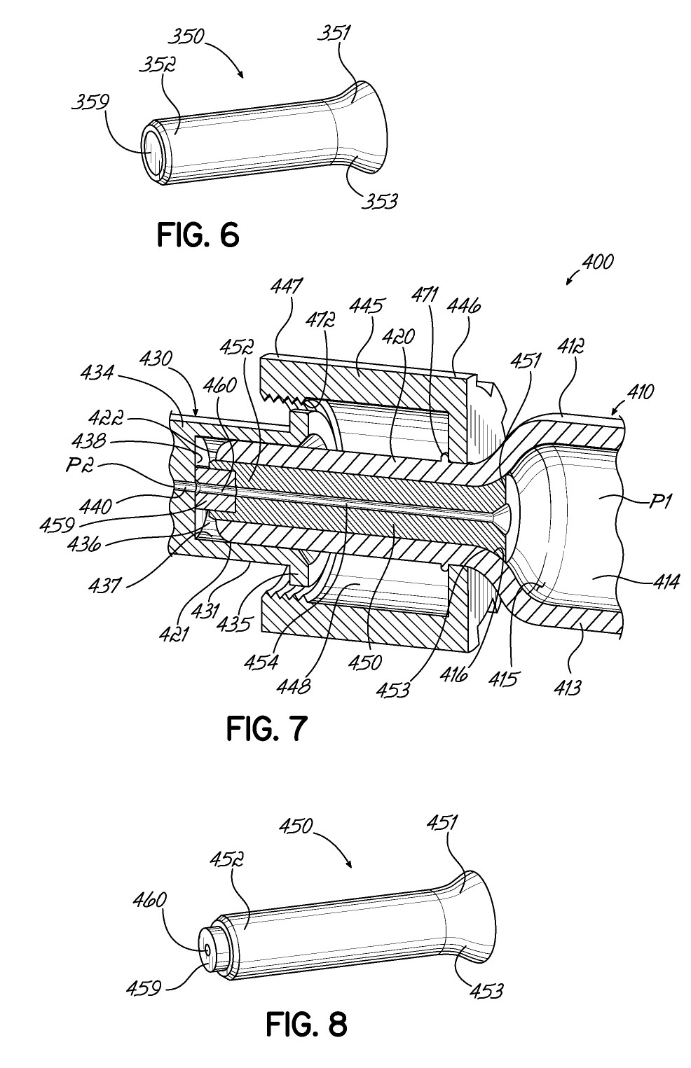

Although the flow control member 250 is shown as defining a lumen 254 that extends therethrough, in other embodiments, a flow control member can define a lumen that does not extend the entire length of the flow control member. Similarly stated, in some embodiments, a flow control member can define a blind lumen. For example, FIGS. 5 and 6 show a portion of a medical injector 300 according to another embodiment. The medical injector 300 includes a medicament container 310, a needle assembly 330 and a flow control member 350. The medicament container 310 is similar to the medicament container 210 shown and described above, and is therefore not described in detail below. The medicament container 310 includes a side wall 313 that defines a lumen 314. The lumen 314 of the medicament container 310 is configured to contain a medicament of the types described herein. A plunger (not shown in FIG. 5) is movably disposed within the lumen 314 of the medicament container 310 such that movement of the plunger results in injection of the medicament from the medicament container 310 (i.e., flow of the medicament through the needle assembly 330).

As shown in FIG. 5, a distal end portion 312 of the medicament container 310 includes a tapered protrusion 320. At least a portion of the protrusion 320 is configured to be disposed within a volume 336 defined by a needle hub 334 when the needle assembly 330 is coupled to the medicament container 310. An outer surface 321 of the protrusion 320 includes a lip or shoulder 371 configured to limit axial movement of a coupler 345 about the protrusion when the coupler 345 is disposed about the protrusion 320.

The needle assembly 330 includes a needle 340 and the hub 334. A proximal end portion 331 of the hub 334 includes a flange 335 and defines the volume 336. The needle 340 (a portion of which is shown in FIG. 5) is coupled to the hub 334 such that a proximal portion of the needle 340 is disposed within the volume 336. Similarly stated, the needle 340 is coupled to the hub 334 such that a proximal portion of the needle 340 extends proximally from an interior end surface 338 of the hub 334.

When the needle assembly 330 is coupled to the medicament container 310, the tapered protrusion 320 of the medicament container 310 is disposed within the volume 336 of the hub 334 such that a portion of the outer surface 321 of the protrusion 320 engages a portion of an inner (i.e., circumferential) surface 337 of the hub 334 to produce a substantially fluid-tight seal. Moreover, when the tapered protrusion 320 of the medicament container 310 is disposed within the volume 336 of the hub 334, a distal end surface 322 of the protrusion 320 is spaced apart from the end surface 338 of the hub 334. In this manner, the engagement between the outer surface 321 of the protrusion 320 and the inner surface 337 of the hub 334 limits the axial movement of the protrusion 320 within the hub 334 in the distal direction. Similarly stated, because the distal end surface 322 of the protrusion 320 does not contact the interior end surface 338 of the hub 334, the protrusion 320 is free to move within the coupler 345 in a distal direction until the outer surface 321 of the protrusion 320 contacts the inner surface 337 of the hub 334.

The coupler 345 includes a distal end portion 347, a proximal end portion 346, and defines a lumen 348 therethrough. The coupler 345 is disposed about the protrusion 320 of the medicament container 310 such that the protrusion 320 is within the lumen 348 of the coupler 345. More specifically, a portion of the proximal end portion 346 of the coupler 345 engages the lip or shoulder 371 of the protrusion 320 such that movement of the coupler 345 axially relative to the medicament container 310 is resisted. A portion of the lumen 348 of the coupler 345 includes female threads 372 configured to receive and/or engage the flange 335 of the hub 334 to threadably couple the hub 334 to the coupler 345, as described above.

The flow control member 350 includes a proximal end portion 351 and a distal end portion 352 and defines a lumen 354. The proximal end portion 351 of the flow control member 350 includes a tapered outer surface 353. The flow control member 350 is disposed within the distal end portion 312 of the medicament container 310 such that the tapered outer surface 353 is engaged with a shoulder 316 defined by an inner surface 315 of the medicament container 310. In this manner, movement of the flow control member 350 within the medicament container 310 can be limited. In some embodiments, a portion of the flow control member 350 can form an interference fit within the protrusion 320 of the medicament container 310. Additionally, the tapered outer surface 353 and the shoulder 316 form a substantially fluid-tight seal. Thus, when the medicament is conveyed from the medicament container 310 to the needle assembly 330, substantially all of the medicament will be conveyed via the lumen 354, as described below.

The distal end portion 352 of the flow control member 350 includes a frangible portion 359 that fluidically isolates the distal end of the lumen 354. Although the frangible portion 359 is shown as being a separate member that is coupled the distal end portion 352 of the flow control member 350, in other embodiments, the frangible portion 359 and the flow control member 350 can be monolithically constructed.

When the needle assembly 330 is coupled to the medicament container 310, the proximal portion of the needle 340 is disposed through the frangible portion 359 and into the lumen 354 of the flow control member 350. Similarly stated, when the needle assembly 330 is coupled to the medicament container 310, the proximal portion of the needle 340 pierces the frangible portion 359 such that the needle 340 is in fluid communication with the lumen 354. Moreover, the proximal portion of the needle 340 and the frangible portion 359 of the flow control member 350 form a substantially fluid-tight seal. Thus, when the medicament is conveyed from the medicament container 310 to the needle assembly 330, substantially all of the medicament will be conveyed via the lumen 354 into the needle 340. In this manner, when the medicament is conveyed from the medicament container 310 to the needle assembly 330, the flow passageway (not shown in FIG. 5) does not include the volume 336 defined by the hub 334.

As described above, the diameter and/or the length of the lumen 354 can be any suitable size to control a flow rate of the medicament from the medicament container 310 to the needle assembly 330. More particularly, the diameter and/or the length of the lumen 354 can be selected to produce a pressure drop when the medicament flows from the medicament container 310 to the needle assembly 330 during an injection event. More particularly, in some embodiments, the flow control member 350 can cause the pressure of the medicament to be reduced from a first pressure P1 at a first location (within the medicament container 310) to a second pressure P2 (within the needle 340). In this manner, the flow control member 350 can control the flow and/or pressure of the medicament within the needle assembly 330 during an injection event.

More particularly, because the proximal portion of the needle 340 and the frangible portion 359 of the flow control member 350 form a substantially fluid-tight seal, the interior end surface 338 of the hub 334 is not exposed to the pressure P2 of the medicament within the needle. In this manner, the flow control member 350 limits the force acting on the hub 334 in the distal direction, thereby reducing the likelihood that the coupler 345 and/or a portion of the hub 334 (e.g., the flange 335) will fail during use. Similarly stated, the arrangement shown in FIG. 5 results in the pressure P2 being exerted only on the cross-sectional area of the needle 340, rather than the interior end surface 338 of the hub 334. Because the cross-sectional area of the needle 340 is less than the area of the interior end surface 338 of the hub 334, the overall distal force acting on the hub 334 is reduced (see e.g., equation (1)).

Although the flow control member 350 is shown and described above as defining a lumen 354 that produces a pressure drop during an injection event, in other embodiments, the lumen 354 can be sized such that the flow control member 350 does not produce a significant pressure drop and/or does not significantly control the flow of the medicament from the medicament container 310 to the needle 340. Similarly stated, in other embodiments, the lumen 354 can be sized such that the pressure P2 is substantially equal to the pressure P1. In such embodiments, the flow control member 350 can control, limit and/or prevent flow of the medicament into the volume 336 of the hub 334 during an injection event.

Although the flow control member 350 and the needle 340 are shown and described above as producing a substantially fluid-tight seal to control, limit and/or prevent flow of the medicament into the volume 336 of the hub 334 during an injection event, in other embodiments, a needle need not extend into the volume 336 of the hub and/or pierce a portion of the flow control member 350. For example, FIGS. 7 and 8 show a portion of a medical injector 400 according to another embodiment. The medical injector 400 includes a medicament container 410, a needle assembly 430 and a seal member 450. The medicament container 410 is similar to the medicament containers 210 and 310 shown and described above, and is therefore not described in detail below. The medicament container 410 includes a side wall 413 that defines a lumen 414. The lumen 414 of the medicament container 410 is configured to contain a medicament of the types described herein. A plunger (not shown in FIG. 5) is movably disposed within the lumen 414 of the medicament container 410 such that movement of the plunger results in injection of the medicament from the medicament container 410 (i.e., flow of the medicament through the needle assembly 430).

As shown in FIG. 7, a distal end portion 412 of the medicament container 410 includes a tapered protrusion 420. At least a portion of the protrusion 420 is configured to be disposed within a volume 436 defined by a needle hub 434 when the needle assembly 430 is coupled to the medicament container 410. An outer surface 421 of the protrusion 420 includes a lip or shoulder 471 configured to limit axial movement of the coupler 445 about the protrusion when the coupler 445 is disposed about the protrusion 420.

The needle assembly 430 includes a needle 440 (a portion of which is shown in FIG. 7) and the hub 434. A proximal end portion 431 of the hub 434 includes a flange 435 and defines the volume 436. The needle 440 is coupled to the hub 434 such that the needle 440 is in fluid communication with the volume 436. Moreover, in contrast to the needle assembly 330 shown and described above, the needle 440 is coupled to the hub 434 such that a proximal portion of the needle 440 is substantially flush with or recessed from an interior end surface 438 of the hub 434.

When the needle assembly 430 is coupled to the medicament container 410, the tapered protrusion 420 of the medicament container 410 is disposed within the volume 436 of the hub 434 such that a portion of the outer surface 421 of the protrusion 420 engages a portion of an inner (i.e., circumferential) surface 437 of the hub 434 to produce a substantially fluid-tight seal. Moreover, when the tapered protrusion 420 of the medicament container 410 is disposed within the volume 436 of the hub 434, a distal end surface 422 of the protrusion 420 is spaced apart from the end surface 438 of the hub 434. Because the distal end surface 422 of the protrusion 420 does not contact the end surface 438 of the hub 434, the protrusion 420 is free to move within the coupler 445 until the outer surface 421 of the protrusion 420 contacts the inner surface 437 of the hub 434. Although the outer surface 421 of the protrusion 420 and the inner surface 437 of the hub 434 are shown and described as being engaged to produce a substantially fluid-tight seal, in other embodiments, the outer surface 421 of the protrusion 420 and the inner surface 437 of the hub 434 need not be engaged or produce a substantially fluid-tight seal.

The coupler 445 includes a distal end portion 447, a proximal end portion 446, and defines a lumen 448 therethrough. The coupler 445 is disposed about the protrusion 420 of the medicament container 410 such that the protrusion 420 is within the lumen 448 of the coupler 445. More specifically, a portion of the proximal end portion 446 of the coupler 445 engages the lip or shoulder 471 of the protrusion 420 such that movement of the coupler 445 axially relative to the medicament container 410 is resisted. A portion of the lumen 448 of the coupler 445 includes female threads 472 configured to receive and/or engage the flange 435 of the hub 434 to threadably couple the hub 434 to the coupler 445, as described above.

The seal member 450 includes a proximal end portion 451 and a distal end portion 452 and defines a lumen 454. The proximal end portion 451 of the seal member 450 includes a tapered outer surface 453. The seal member 450 is disposed within the distal end portion 412 of the medicament container 410 such that the tapered outer surface 453 is engaged with a shoulder 416 defined by an inner surface 415 of the medicament container 410. In this manner, movement of the seal member 450 within the medicament container 410 can be limited. Additionally, the tapered outer surface 453 and the shoulder 416 form a substantially fluid-tight seal. Thus, when the medicament is conveyed from the medicament container 410 to the needle assembly 430, substantially all of the medicament will be conveyed via the lumen 454.

The distal end portion 452 of the seal member 450 includes a flexible portion 459 that defines a lumen 460 that is substantially coaxial with the lumen 454. As shown in FIG. 7, when the seal member 450 is disposed within the medicament container 410, at least a portion of the flexible portion 459 is disposed outside of the protrusion 420 of the medicament container 410. Similarly stated, when the seal member 450 is disposed within the medicament container 410, an end surface of the flexible portion 459 is disposed distally from the end surface 422 of the protrusion 420 of the medicament container 410.

When the needle assembly 430 is coupled to the medicament container 410, the end surface of the flexible portion 459 contacts a portion of the interior end surface 438 of the hub 434 to form a substantially fluid-tight seal. Thus, when the medicament is conveyed from the medicament container 410 to the needle assembly 430, the flow passageway (not shown in FIG. 7) does not include the volume 436 defined by the hub 434. Similarly stated, when the medicament is injected from the medicament container, the interior end surface 438 of the hub 434 is not exposed to the pressure P2 of the medicament within the needle 440. In this manner, the seal member 450 limits the force acting on the hub 434 in the distal direction during an injection event by reducing the area of the hub 434 exposed to the injection pressure P2.

When the needle assembly 430 is coupled to the medicament container 410, the flexible portion 459 deforms in a longitudinal direction (the deformation is not shown in FIG. 7). The deformation of the flexible portion 459 allows the outer surface 421 of the protrusion 420 and the inner surface 437 of the hub 434 to be fully engaged to form a substantially fluid-tight seal. Additionally, the deformation of the flexible portion 459 produces force against the interior end surface 438 that enhances the integrity of the substantially fluid-tight seal between the end surface of the flexible portion 459 and the interior end surface 438 of the hub 434.

In addition to producing a fluid-tight seal, the diameter and/or the length of the lumen 454 of the seal member 450 can be any suitable size to control a flow rate of the medicament from the medicament container 410 to the needle assembly 430. More particularly, as described above, the diameter and/or the length of the lumen 454 and the lumen 460 can be selected to produce a pressure drop when the medicament flows from the medicament container 410 to the needle assembly 430 during an injection event. More particularly, in some embodiments, the seal member 450 can cause the pressure of the medicament to be reduced from a first pressure P1 at a first location (within the medicament container 410) to the second pressure P2 at a second location (within the needle 440). In this manner, the seal member 450 can control the flow and/or pressure of the medicament within the needle assembly 430 during an injection event.

The flexible portion 459 can be constructed from any suitable material formulated to provide the desired flexibility and produce a fluid-tight seal when in contact with the hub 434. In some embodiments, the flexible portion 459 can deform elastically when the needle assembly 430 is coupled to the medicament container 410. In other embodiments, the flexible portion 459 can deform plastically when the needle assembly 430 is coupled to the medicament container 410.

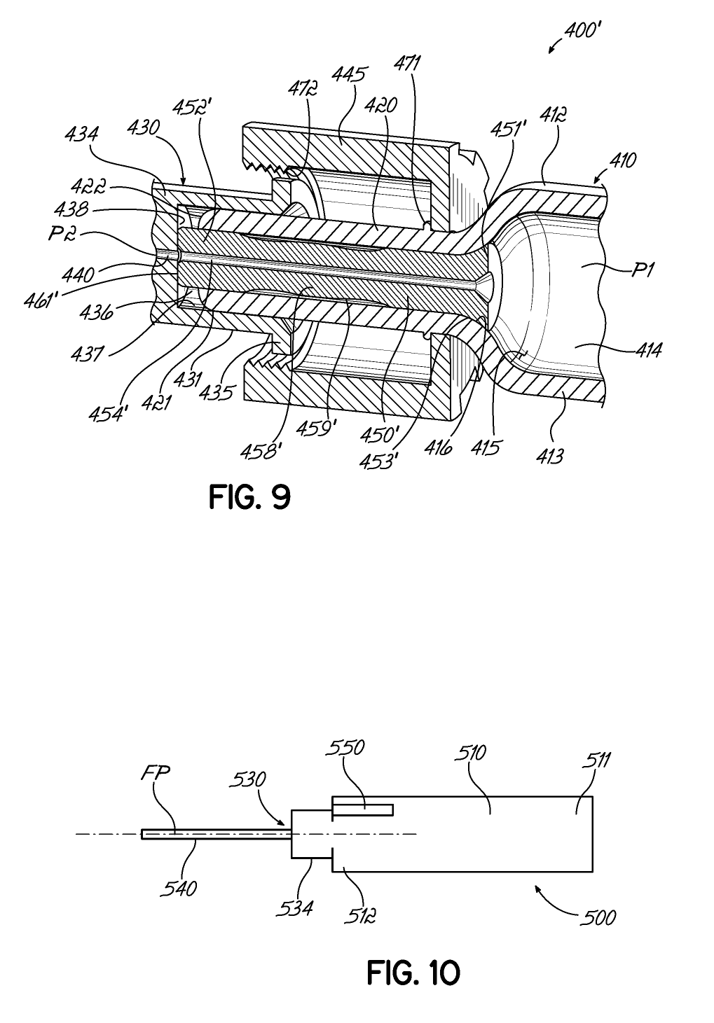

Although the flexible portion 459 is shown as being a separate member that is coupled the distal end portion 452 of the seal member 450, in other embodiments, the flexible portion 459 and the seal member 450 can be monolithically constructed. In other embodiments, a flexible portion need not be disposed at a distal end of a seal member, but can be disposed at any longitudinal position of a seal member. For example, FIG. 9 shows a portion of medical injector 400′ according to another embodiment. The medical injector 400′ includes a medicament container 410, a needle assembly 430 and a seal member 450′. The medicament container 410 and the needle assembly 430 are described above with reference to FIG. 7, and are therefore not described in detail below.

The seal member 450′ includes a proximal end portion 451′, a distal end portion 452′ and a central portion 458′, and defines a lumen 454′ therethrough. The proximal end portion 451′ of the seal member 450′ includes a tapered outer surface 453′. The seal member 450′ is disposed within the distal end portion 412 of the medicament container 410, as described above.

As shown in FIG. 9, when the seal member 450′ is disposed within the medicament container 410, the distal end portion 452′ is disposed outside of the protrusion 420 of the medicament container 410. Similarly stated, when the seal member 450′ is disposed within the medicament container 410, a distal end surface 461′ of the seal member 450′ is disposed distally from the distal end surface 422 of the protrusion 420 of the medicament container 410. When the needle assembly 430 is coupled to the medicament container 410, the distal end surface 461′ of the seal member 450′ contacts a portion of the interior end surface 438 of the hub 434 to form a substantially fluid-tight seal. Thus, when the medicament is conveyed from the medicament container 410 to the needle assembly 430, the flow passageway (not shown in FIG. 9) does not include the volume 436 defined by the hub 434.

The central portion 458′ of the seal member 450′ includes a flexible portion 459′. When the needle assembly 430 is coupled to the medicament container 410, the flexible portion 459′ deforms in a longitudinal direction (the deformation is not shown in FIG. 9). The deformation of the flexible portion 459′ allows the outer surface 421 of the protrusion 420 and the inner surface 437 of the hub 434 to be fully engaged to form a substantially fluid-tight seal. Additionally, the deformation of the flexible portion 459′ produces force against the interior end surface 438 that enhances the integrity of the substantially fluid-tight seal between the distal end surface 461′ of the seal member 450′ and the interior end surface 438 of the hub 434.

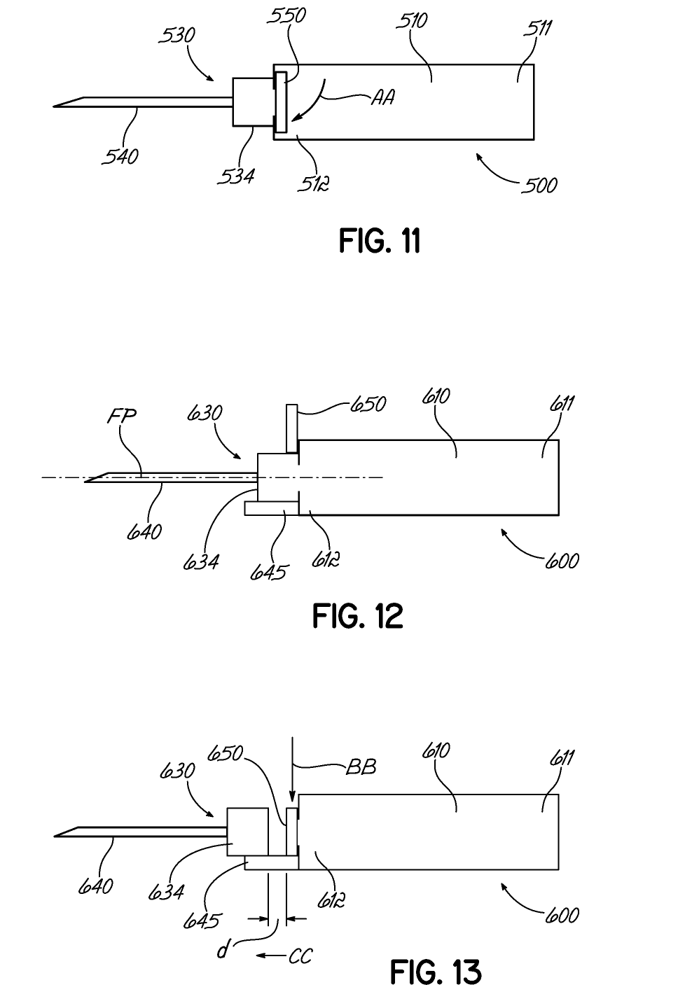

FIGS. 10 and 11 are schematic illustrations of a medical injector 500 according to another embodiment, in a first configuration and a second configuration, respectively. The medical injector 500 includes a medicament container 510, a needle assembly 530 and a flow control member 550. The medicament container 510 has a proximal end portion 511 and a distal end portion 512. The medicament container 510 is configured to contain a medicament such as the types described herein. In some embodiments, the medicament container 510 can include a plunger (not shown in FIGS. 10 and 11) configured to displace the medicament within the medicament container 510.

The needle assembly 530 includes a needle 540 and a hub 534. The needle 540 can be, for example, a small-bore needle (e.g., a needle having a bore size less than or equal to that of a 27 gauge needle) configured to convey the medicament from the medicament container 510 to the patient's body. The needle 540 and the hub 534 are collectively coupled to the distal end portion 512 of the medicament container 510 such that the needle 540 is in fluid communication with the medicament container 510. In this manner, when the medicament is injected from the medicament container 510, the medicament is conveyed from the medicament container 510 through the needle assembly 530 along a medicament flow passageway FP (shown as a dashed line in FIG. 10). The needle assembly 530 can be coupled to the medicament container 510 by any suitable coupler (e.g., a Luer connector) that provides a substantially fluid-tight seal (i.e., a seal that that substantially prevents a liquid and/or a gas from passing therethrough) between the needle assembly 530 and the medicament container 510. Moreover, such a coupler can be configured to maintain a position of the needle assembly 530 relative to the distal end 512 of the medicament container 510.

The flow control member 550 is disposed within the distal end portion 512 of the medicament container 510 and is configured to move between a first position (FIG. 10) and a second position (FIG. 11). When the flow control member 550 is in the first position, the needle assembly 530 is in fluid communication with the medicament container 510. Similarly stated, when the flow control member 550 is in the first position, the medicament can be conveyed from the medicament container 510 through the needle assembly 530 along a medicament flow passageway FP.

The flow control member 550 can move from the first position to the second position, as shown by the arrow AA in FIG. 11. When the flow control member 550 is in the second position, the needle assembly 530 is fluidically isolated from the medicament container 510. Similarly stated, when the flow control member 550 is in the second position, the medicament flow passageway FP is blocked such that the medicament cannot be conveyed from the medicament container 510 to the needle assembly 530.

Although the flow control member 550 is shown and described as fluidically isolating the needle assembly 530 and the medicament container 510 when in the second position, in other embodiments, the flow control member 550 can control a pressure and/or a flow rate of the medicament along the flow passageway FP when the flow control member 550 is in the second position. In some embodiments, for example, the flow control member 550 can produce a pressure drop along and/or within the flow passageway FP when in the flow control member 550 is in the second position. For example, in some embodiments, the flow control member 550 can define an orifice (not shown) configured to produce a pressure drop within the flow passageway FP when the flow control member 550 is in the second position.

Although the flow control member 550 is shown and described as being disposed within the medicament container 510, in other embodiments, a flow control member can be disposed outside of a medicament container. For example, FIGS. 12 and 13 are schematic illustrations of a medical injector 600 according to another embodiment, in a first configuration and a second configuration, respectively. The medical injector 600 includes a medicament container 610, a needle assembly 630, a coupler 645 and a flow control member 650. The medicament container 610 has a proximal end portion 611 and a distal end portion 612. The medicament container 610 is configured to contain a medicament such as the types described herein. In some embodiments, the medicament container 610 can include a plunger (not shown in FIGS. 12 and 13) configured to displace the medicament within the medicament container 610.

The needle assembly 630 includes a needle 640 and a hub 634. The needle 640 can be, for example, a small-bore needle (e.g., a needle having a bore size less than or equal to that of a 27 gauge needle) configured to convey the medicament from the medicament container 610 to the patient's body. The needle 640 and the hub 634 are collectively coupled to the distal end portion 612 of the medicament container 610 by the coupler 645 such that the needle 640 is in fluid communication with the medicament container 610. In this manner, when the medicament is injected from the medicament container 610, the medicament is conveyed from the medicament container 610 through the needle assembly 630 along a medicament flow passageway FP (shown as a dashed line in FIG. 12).

The needle assembly 630 is coupled to the medicament container 610 by the coupler 645. The coupler 645 can be any suitable coupler (e.g., a Luer connector) that provides a substantially fluid-tight seal (i.e., a seal that that substantially prevents a liquid and/or a gas from passing therethrough) between the needle assembly 630 and the medicament container 610. Moreover, the coupler 645 is configured to maintain a position of the needle assembly 630 relative to the distal end portion 612 of the medicament container 610.

The flow control member 650 is coupled to the distal end portion 612 of the medicament container 610 and is configured to move between a first position (FIG. 12) and a second position (FIG. 13). When the flow control member 650 is in the first position, the needle assembly 630 is in fluid communication with the medicament container 610. Similarly stated, when the flow control member 650 is in the first position, the medicament can be conveyed from the medicament container 610 through the needle assembly 630 along a medicament flow passageway FP.

In certain instances, the pressure of the medicament in the medicament container 610 can cause the coupler 645 and/or a portion of the needle assembly 630 to fail such that the needle assembly 630 detaches from the medicament container 610 and moves, for example, a distance d substantially in the direction CC, as shown in FIG. 13. Said another way, in some instances, the coupler 645 can fail to maintain the position of the needle assembly 630 relative to the medicament container 610 during an injection event. When such detachment occurs, the flow control member 650 is configured to fluidically isolate the medicament container 610 from the needle assembly 630. More particularly, when such detachment occurs, the flow control member 650 is configured to move between the first position and the second position, as shown by the arrow BB in FIG. 13 to block the medicament flow passageway FP. In this manner, when detachment of the needle assembly 630 from the medicament container 610 occurs, the flow control member 650 prevents further conveyance of the medicament and/or limits the pressure of the medicament within the needle assembly 630. Similarly stated, the flow control member 650 prevents the needle assembly 630 from moving relative to the medicament container 610 greater than the distance d.

The flow control member 650 can moved by any suitable mechanism. For example, in some embodiments, the movement of the needle assembly 630 relative to the coupler 645 can release a biasing member, such as, for example, a spring, thereby moving the flow control member 650.

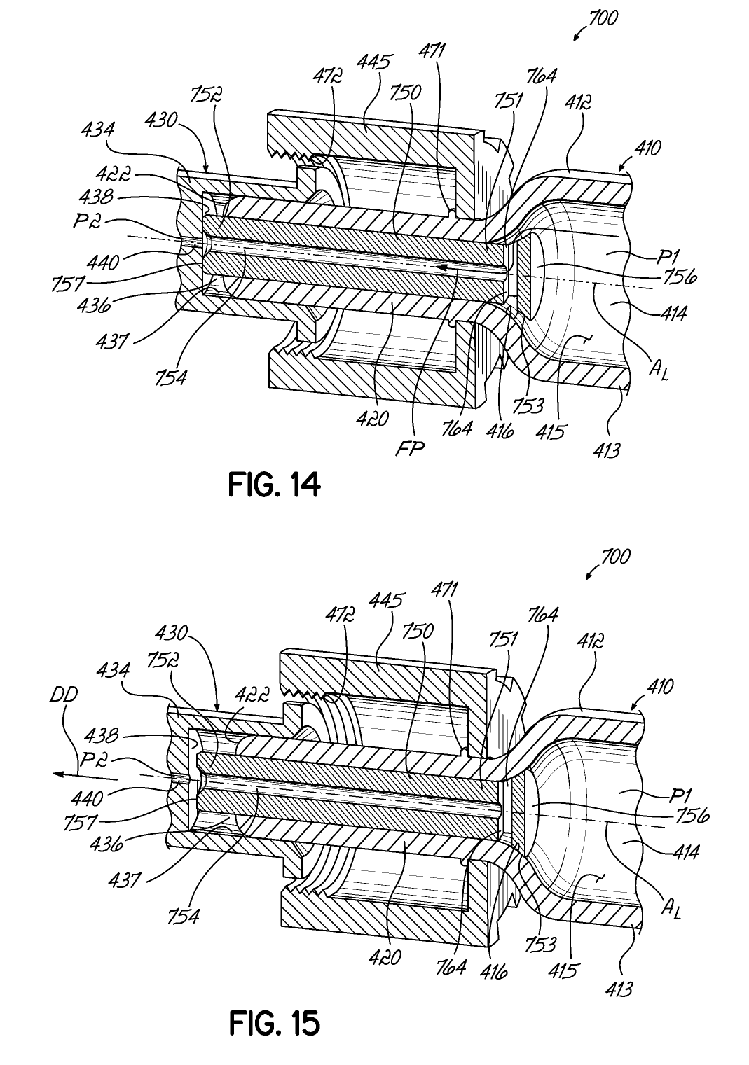

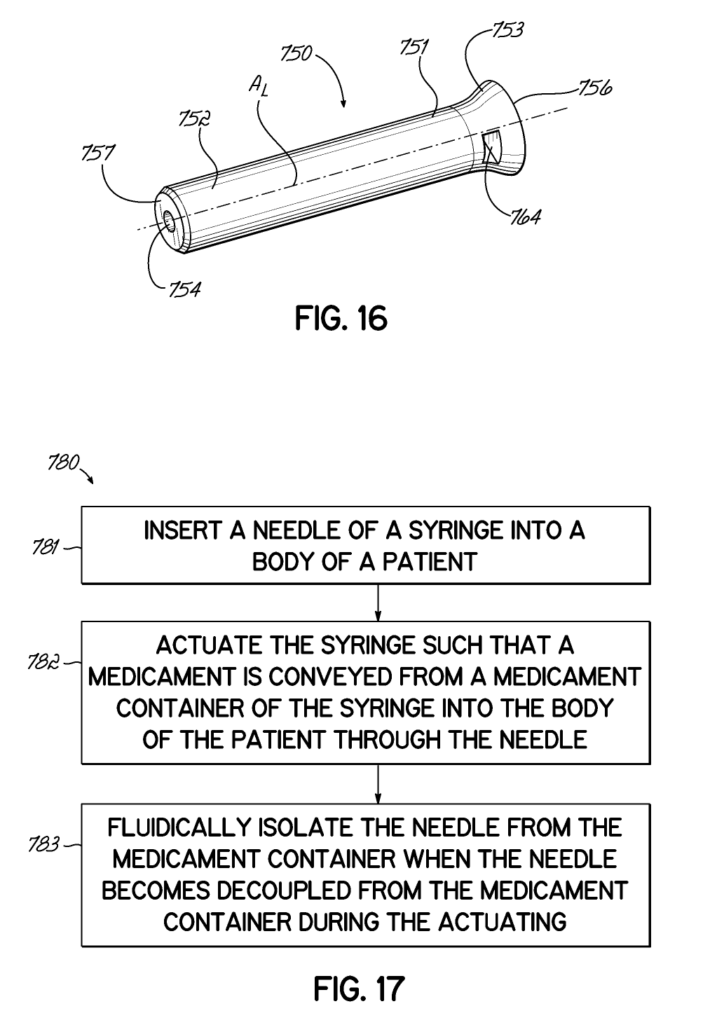

FIGS. 14-16 show a portion of a medical injector 700 according to another embodiment. FIG. 14 shows the portion of the medical injector 700 in a first configuration. FIG. 15 shows the portion of the medical injector 700 in a second configuration. FIG. 16 shows a flow control member 750 of the medical injector 700. The medical injector 700 includes a medicament container 410, a needle assembly 430, a coupler 445 and the flow control member 750. The medicament container 410, the needle assembly 430 and the coupler 445 are described above with reference to FIG. 7, and are therefore not described in detail below.

The flow control member 750 includes a proximal end portion 751 and a distal end portion 752, and defines a lumen 754 that is substantially parallel to a longitudinal axis AL of the flow control member 750. As shown in FIGS. 14 and 15, the lumen 754 is sized larger than the corresponding lumen 454′ in the flow control member 450′ of the previously-described embodiment shown in FIG. 9. More particularly, the lumen in the flow control member may be approximately the same size as the needle in the needle hub (as shown in FIG. 9), or the lumen may be configured to be larger, such as 1.5 times the size of the needle (as shown in FIGS. 14 and 15). To this end, the respective sizes of the lumen in the flow control member may be adjusted to balance the considerations of the required force to actuate flow through the medical injector (which increases as the size of the lumen decreases) and the decoupling force applied to the needle assembly by the flow of medicament (which decreases as the size of the lumen decreases).

The distal end portion 752 of the flow control member 750 includes a distal end surface 757. The proximal end portion 751 of the flow control member 750 includes a tapered outer surface 753 and a proximal end surface 756. The proximal end portion 751 also defines two lateral flow passages 764 in fluid communication with the lumen 754. Thus, as described in more detail below, the lateral flow passages 764 can place the lumen 414 of the medicament container 410 in fluid communication with the lumen 754 of the flow control member 750.

The flow control member 750 is movably disposed within the distal end portion 412 of the medicament container 410. More particularly, the flow control member 750 is configured to move along the longitudinal axis AL between a first position (FIG. 14) and a second position (FIG. 15). When the flow control member 750 is in the first position and when the needle assembly 430 is coupled to the medicament container 410, the tapered outer surface 753 is spaced apart from the shoulder 416 defined by an inner surface 415 of the medicament container 410. In this manner, the lateral flow passages 764 are in fluid communication with the lumen 414 of the medicament container 410. Thus, when the flow control member 750 is in the first position, the medicament can be conveyed from the medicament container 410 through the lumen 754 of the flow control member 750 via a medicament flow passageway FP.

When the flow control member 750 is in the first position and when the needle assembly 430 is coupled to the medicament container 410, at least a portion of the distal end portion 752 is disposed outside of the protrusion 420 of the medicament container 410 such that a distal end surface 757 of the flow control member 750 contacts a portion of the interior end surface 438 of the hub 434 to form a substantially fluid-tight seal. Thus, when the medicament is conveyed from the medicament container 410 to the needle assembly 430, the flow passageway FP does not include the volume 436 defined by the hub 434. Similarly stated, when the medicament is injected from the medicament container 410, the interior end surface 438 of the hub 434 is not exposed to a pressure P2 of the medicament within the needle 440. In this manner, the flow control member 750 limits the force acting on the hub 434 in the distal direction during an injection event by reducing the area of the hub 434 exposed to the injection pressure P2. In some embodiments, a portion of the flow control member 750 can deform when the flow control member 750 is in the first position and when the needle assembly 430 is coupled to the medicament container 410, as described above.

In addition to producing a fluid-tight seal, the diameter and/or the length of the lumen 754 and/or the lateral flow passages 764 of the flow control member 750 can be any suitable size to control a flow rate of the medicament from the medicament container 410 to the needle assembly 430. More particularly, as described above, the diameter and/or the length of the lumen 754 and/or the lateral flow passages 764 can be selected to produce a pressure drop when the medicament flows from the medicament container 410 to the needle assembly 430 during an injection event. More particularly, in some embodiments, the flow control member 750 can cause the pressure of the medicament to be reduced from a first pressure P1 (within the medicament container 410) to the second pressure P2 (within the needle 440), which is less than the first pressure P1. In this manner, the flow control member 750 can control the flow and/or pressure of the medicament within the needle assembly 430 during an injection event.

In certain instances, the pressure P1 of the medicament in the medicament container 410 may cause the coupler 445 and/or a portion of the needle assembly 430 to fail such that the needle assembly 430 becomes detached from the medicament container 410. In such instances the needle assembly 430 can move distally relative to the medicament container 410, as shown by the arrow DD in FIG. 15. Said another way, in some instances, the coupler 445 may fail to maintain the position of the needle assembly 430 relative to the medicament container 410 during an injection event. If and when such distal movement of the needle assembly 430 occurs, the flow control member 750 is configured to move from its first position (FIG. 14) to its second position (FIG. 15). More particularly, when such movement of the needle assembly 430 occurs, the distal end surface 757 is spaced apart from the interior end surface 438 of the hub 434, thereby allowing distal movement of the flow control member 750. Thus, the force of the pressure P1 acting on the proximal end surface 756 of the flow control member 750 moves the flow control member 750 distally to its second position.

When the flow control member 750 is in the second position, the tapered outer surface 753 is engaged with the shoulder 416 defined by an inner surface 415 of the medicament container 410. In this manner, further distal movement of the flow control member 750 within the medicament container 410 is limited. Additionally, the tapered outer surface 453 and the shoulder 416 form a substantially fluid-tight seal. Thus, the lateral flow passages 764 are fluidically isolated from the lumen 414 of the medicament container 410. Similarly stated, when the flow control member 750 is in the second position the medicament flow passageway FP is blocked such that medicament cannot be conveyed from the medicament container 410 to the needle assembly 430.

FIG. 17 is a flow chart of a method 780 of using a medical injector according to one embodiment. The medical injector includes a needle and a syringe. The method includes inserting the needle into a body of a patient, at step 781. In some embodiments, the inserting includes inserting the needle of the syringe into a skin of the body of the patient to a depth of at least 1 millimeters. In other embodiments, such as, for example, an injection associated with mesotherapy, the needle can be inserted into the skin to a depth of between 1 millimeters and 2 millimeters. In yet other embodiments, such as, for example, an injection associated with intradermal therapy, the needle can be inserted into the skin to a depth of between 2 millimeters and 5 millimeters. In yet other embodiments, such as, for example, an injection associated with subdermal therapy, the needle can be inserted into the skin to a depth greater than 5 millimeters.

The method includes actuating the syringe such that a medicament is conveyed from a medicament container of the syringe into the body of the patient through the needle, at step 782. The medicament can be, for example, a dermal filler, a sub-dermal filler, a therapeutic substance for mesotherapy or the like. In some embodiments, the actuating includes applying a compressed gas to a proximal end of the medicament container of the syringe and moving a plunger within the medicament container such that a pressure of the medicament within the medicament container is greater than 70 p.s.i.. It will be understood that the pressure of the medicament within the medicament container may also be less than 70 p.s.i. in alternative embodiments. In some embodiments, the actuating includes actuating a machine (e.g., a stepper motor) that actuates the medicament container of the syringe.

The method includes fluidically isolating the needle from the medicament container when the needle becomes decoupled from the medicament container during the actuation, at step 783. The needle can be fluidically isolated from the medicament container via any of the methods described above. In some embodiments, the needle can be fluidically isolated from the medicament container by a flow control member disposed, at least partially, within the medicament container.

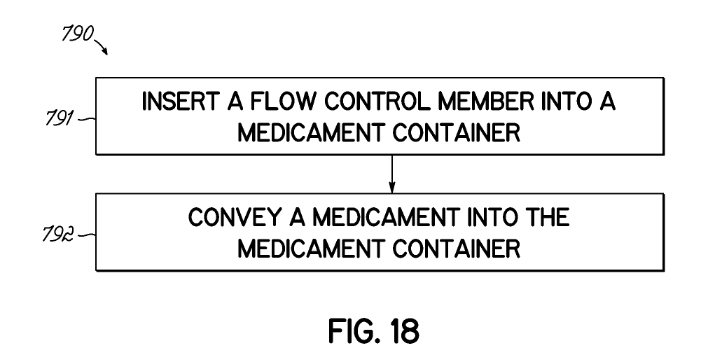

FIG. 18 is a flow chart of a method 790 of filling a medicament container according to another embodiment. The method includes inserting a flow control member into the medicament container, at step 791. The medicament container can be any commercially-available syringe, vial, ampoule, cartridge or the like, such as those shown and described above. The flow control member, which can be any of the flow control members and/or sealing members shown and described above, is inserted into the medicament container from a proximal end. In some embodiments, the flow control member can be moved within the medicament container until a tapered portion of the flow control member contacts a shoulder or protrusion within the medicament container.

A medicament is then conveyed into the medicament container, at step 792. The medicament can be any dermal filler, a sub-dermal filler or therapeutic substance of the types described herein. The medicament is conveyed into the medicament container from the proximal end. In some embodiments, the medicament can exert a force on the flow control member when the medicament is being conveyed into the medicament container, which can move the flow control member within the medicament container until the tapered portion of the flow control member contacts the shoulder or protrusion within the medicament container. Similarly stated, in some embodiments, the operation of conveying the medicament into the medicament container can cause the flow control member to be seated (i.e., oriented and/or positioned) within the medicament container. The medicament container can then be delivered to a practitioner for use in the manner described above.

The methods and apparatus described herein provide a flow and/or pressure control mechanism for commercially-available medicament containers (e.g., syringes, vials, ampoules, cartridges or the like) without modification of the medicament container and/or the needle assembly. Similarly stated, the flow control members and/or sealing members described herein can be used with commercially-available syringes without modifying the syringe and/or the needle assembly.

The methods and apparatus described herein provide control of the flow rate and/or pressure of a medicament during injection at the distal end of a medical injector. Said another way, the methods and apparatus described herein provide control of the flow rate and/or pressure of a medicament during injection at the delivery end of the medical injector (i.e., adjacent the needle). Similarly stated, the methods and apparatus described herein can control of the flow rate and/or pressure of a medicament by acting directly on the medicament, rather than by acting on an actuator that provides the force and/or pressure for injecting the medicament. Said another way, the methods and apparatus described herein can control of the flow rate and/or pressure of a medicament by acting directly on the medicament, rather than by controlling or limiting the force produced by an actuator disposed at a proximal end of the medicament container. Such an arrangement can provide more accurate control and/or improved transient control.

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

Although the needles are shown and described above as generally being hypodermic needles, in other embodiments, a needle can include any suitable injection member, such as a cannula, trocar, or the like, configured to convey a convey a medicament to the patient's body. For example, in some embodiments, a needle can include a relatively large-bore trocar configured to puncture the skin and convey a medicament therein. In some embodiments, for example, a needle can include an 18 gauge or larger trocar. In other embodiments, a needle includes a relatively short delivery member configured to be coupled to a jet injector. Such delivery members can be, for example, relatively small-bore needles configured to make a small incision. Such delivery members can have a length of less than 5 millimeters.

Although embodiments above describe a medicament container made of glass, in some embodiments, the medicament container can be made of plastic, metal or any other suitable material. In some embodiments, for example, the medicament container can be made of glass having a polymer coating on the surface of the medicament container. In yet other embodiments, the medicament container can be made of any suitable clear, opaque, translucent and/or rigid material.

Although the flow control members are shown and described above as defining a lumen, in other embodiments, a flow control member can defining multiple lumens. Moreover, in some embodiments, the longitudinal axis of the lumen can be nonparallel to a longitudinal axis of the flow control member and/or a medicament container.

Although the flow control member 550 is shown as rotating within the medicament container 510 when moving between the first position and the second position, in other embodiments, the flow control member 550 can move in any suitable fashion when moving between the first position and the second position. In some embodiments, for example, a flow control member can translate within a medicament container when moving between a first position and a second position. More particularly, in some embodiments, a flow control member can translate in any suitable direction (e.g., along a longitudinal axis of the medicament container, substantially normal to the longitudinal axis of the medicament container, or any direction therebetween) when moving between a first position and a second position.

Although the flow control member 550 is shown as having substantially the same size when in both the first position and the second position, in other embodiments, the flow control member 550 can change size, shape and/or orientation when moving between the first position and the second position.

Although various embodiments have been described as having particular features and/or combinations of components, other embodiments are possible having a combination of any features and/or components from any of embodiments where appropriate. For example, in some embodiments, a medical injector can include a flow control member similar to the flow control member 750 shown and described above with reference to FIGS. 14 -16 and having a sealing portion similar to the sealing portion 459 shown and described above with reference to FIGS. 7 and 8.

While the present invention has been illustrated by a description of various preferred embodiments and while these embodiments have been described in some detail, it is not the intention of the Applicants to restrict or in any way limit the scope of the appended claims to such detail. Additional advantages and modifications will readily appear to those skilled in the art. The various features discussed herein may be used alone or in any combination depending on the needs and preferences of the user. This has been a description of illustrative aspects and embodiments the present invention, along with the preferred methods of practicing the present invention as currently known. However, the invention itself should only be defined by the appended claims.

What is claimed is:

1. A medical injector configured to deliver a medicament to a patient, comprising:
a medicament container including a main lumen, a reduced-diameter protrusion extending distally from the main lumen and including a distal end surface, and an internal shoulder between the main lumen and the protrusion, the medicament container configured to contain and dispense the medicament;
a needle assembly coupled to the protrusion and including a needle configured to receive dispensed medicament from the medicament container; and
a flow control member positioned at least partially within the protrusion and including a distal end portion positioned distal to the distal end surface of the protrusion, the flow control member also including a flow control lumen configured to control at least one of a pressure or a flow of the medicament from the main lumen into the needle assembly, to thereby reduce decoupling forces applied to the needle assembly by the flow of the medicament,
wherein the needle assembly further includes a needle hub engaged with the protrusion of the medicament container, the needle hub including an interior end surface spaced from the distal end surface of the protrusion, and wherein the distal end portion of the flow control member is engaged with the interior end surface of the needle hub to reduce an effective area of the needle hub upon which the medicament applies decoupling forces.

2. The medical injector of claim 1, wherein the flow control member includes a proximal end portion with a tapered outer surface configured to sealingly engage the internal shoulder of the medicament container.

3. A medical injector of claim 1 configured to deliver a medicament to a patient, comprising:
a medicament container including a main lumen, a reduced-diameter protrusion extending distally from the main lumen, and an internal shoulder between the main lumen and the protrusion, the medicament container configured to contain and dispense the medicament;
a needle assembly coupled to the protrusion and including a needle configured to receive dispensed medicament from the medicament container; and
a flow control member positioned at least partially within the protrusion and including a flow control lumen configured to control at least one of a pressure or a flow of the medicament from the main lumen into the needle assembly, to thereby reduce decoupling forces applied to the needle assembly by the flow of the medicament,
wherein the protrusion of the medicament container includes a distal end surface, and the flow control member includes a distal end portion positioned proximal to the distal end surface of the protrusion.

4. The medical injector of claim 3, wherein the flow control member further includes a frangible portion closing off the flow control lumen at the distal end portion, the frangible portion configured to be pierced by the needle to place the flow control lumen in fluid communication with the needle assembly.

5. (canceled)

6. (canceled)

7. The medical injector of claim 1, wherein the flow control member includes a proximal end portion with a tapered outer surface configured to sealingly engage the internal shoulder of the medicament container, and wherein the flow control member includes a flexible portion configured to compress longitudinally to ensure that the distal end portion of the flow control member is engaged with the interior end surface of the needle hub when the tapered outer surface of the flow control member engages the internal shoulder of the medicament container.

8. The medical injector of claim 7, wherein the flexible portion is positioned at the distal end portion of the flow control member.

9. The medical injector of claim 7, wherein the flexible portion is positioned between the proximal end portion and the distal end portion of the flow control member.

10. The medical injector of claim 1, wherein the flow control member is configured to move between a first position in which the medicament can flow from the main lumen into the needle assembly, and a second position in which the flow control member blocks flow of the medicament from the medicament container.

11. A medical injector configured to deliver a medicament to a patient, comprising:
a medicament container including a main lumen, a reduced-diameter protrusion extending distally from the main lumen, and an internal shoulder between the main lumen and the protrusion, the medicament container configured to contain and dispense the medicament;
a needle assembly coupled to the protrusion and including a needle configured to receive dispensed medicament from the medicament container; and
a flow control member positioned adjacent the protrusion and configured to move from a first position to a second position when forces applied by the medicament to the needle assembly decouple the needle assembly and the protrusion,
wherein in the first position, medicament may flow from the main lumen to the needle assembly, and in the second position the flow control member blocks flow of medicament from the medicament container.

12. The medical injector of claim 11, wherein the flow control member is mounted outside the medicament container and is spring-biased to move from the first position to the second position when the needle assembly decouples from the protrusion.

13. The medical injector of claim 11, wherein the flow control member is positioned at least partially within the protrusion, and the flow control member includes a proximal end surface, lateral flow passages located distally of the proximal end surface and in fluid communication with the main lumen, and a flow control lumen in fluid communication with the lateral flow passages and the needle assembly.

14. The medical injector of claim 13, wherein the flow control member further includes a distal end portion engaged with the needle assembly in the first position such that the needle assembly prevents movement of the flow control member in a distal direction.

15. The medical injector of claim 14, wherein when the needle assembly decouples from the protrusion by moving in a distal direction under forces generated by flow of the medicament, the distal end portion of the flow control member is unblocked such that the medicament in the main lumen forces the flow control member to move in a distal direction to the second position, and wherein the lateral flow passages are sealingly blocked by the shoulder of the protrusion in the second position.

16. A method of dispensing medicament into a patient from a medical injector including a medicament container with a main lumen and a reduced-diameter protrusion extending distally from the main lumen, a needle assembly with a needle, and a flow control member disposed at least partially within the protrusion, the method comprising:
inserting the needle into the patient;
actuating a flow of the medicament from the medicament container to the needle assembly;
applying forces to the needle assembly with the medicament until the needle assembly decouples from the protrusion; and
moving the flow control member from a first position in which medicament may flow from the main lumen to the needle assembly to a second position in which the flow control member blocks the flow of the medicament from the medicament container upon decoupling of the needle assembly and the protrusion,
wherein the flow of the medicament tends to bias the flow control member towards the second position in which the flow of the medicament is blocked.

17. The method of claim 16, wherein the flow control member includes a flow control lumen, and the method further comprises:
controlling a pressure or the flow of the medicament through the needle assembly with the flow control lumen.

18. The medical injector of claim 3, wherein the flow control member is configured to move between a first position in which the medicament can flow from the main lumen into the needle assembly, and a second position in which the flow control member blocks flow of the medicament from the medicament container.
